(19) United States
(12) Patent Application Publication
(10) Pub. No.: US 2019/0074094 A1
(43) Pub. Date: Mar. 7, 2019

(54) SYSTEM AND METHOD OF COLLABORATING MEDICAL DATA OVER A WIRELESS NETWORK

(71) Applicants: Giannie Castellanos, Miami Lakes, FL (US); Genny Castellanos, Miami Lakes, FL (US)

(72) Inventors: Giannie Castellanos, Miami Lakes, FL (US); Genny Castellanos, Miami Lakes, FL (US)

(21) Appl. No.: 16/177,506

(22) Filed: Nov. 1, 2018

Related U.S. Application Data
(60) Provisional application No. 62/532,746, filed on Jul. 14, 2017.

(51) Int. Cl. G16H 80/00; G06F 21/62; G16H 40/20; H04L 12/58; H04L 29/08

(52) U.S. Cl. CPC G16H 80/00; G06F 21/6245; G06F 21/6227; G06Q 40/08; H04L 51/24; H04L 67/10; G16H 40/20

(57) ABSTRACT

The present invention discloses a system for the collaborative sharing and scheduling of medical information over a network. A microprocessor is electrically connected to a memory module configured to receive and store transmitted medical information corresponding to a patient referred to a pre-determined medical center. The memory module is wirelessly connected to a secure server for receiving and collaborating patient data information to a plurality of subscribed medical centers. A database is configured to share medical data amongst users who are administratively controlled to facilitate scheduling and referrals of patients.

### CROSS-REFERENCE TO RELATED APPLICATIONS

This application claims priority to U.S. Provisional Application 62/532,746 filed Jul. 14, 2017, entitled “A SYSTEM AND METHOD OF COLLABORATING MEDICAL DATA OVER A WIRELESS NETWORK,” which is hereby incorporated by reference.

### TECHNICAL FIELD

The present invention relates to a system and method of scheduling and collaborating medical information over a network, and more particularly, a system and method of collaborating patient medical data between a plurality of wirelessly connected medical centers.

### BACKGROUND

The exchange of healthcare information allows health care professionals and patients to appropriately access and securely share sensitive information. In recent years, the electronic sharing of healthcare information has increased dramatically bringing about a variety of challenges. This growth necessitates improvements in the quality, safety, and efficiency of information delivery.

The needs and requirements for the exchange of health information differ between practices and states. There is a need for a streamlined platform to safely, accurately, and efficiently share patient information. Antiquated forms of data storage and retrieval such as hard-copies of medical records is no longer the safest or most efficient means of transferring information.

In the current arts, advances in the virtual healthcare setting are increasing. However, a need exists for an easy to operate platform wherein healthcare information can be stored and transmitted without the need for expensive computer systems and software implements. One such improvement is described in the embodiments provided herein.

### SUMMARY OF THE INVENTION

This summary is provided to introduce a variety of concepts in a simplified form that are further disclosed in the detailed description of the invention. This summary is not intended to identify key or essential inventive concepts of the claimed subject matter, nor is it intended for determining the scope of the claimed subject matter.

The embodiments provide a system for collecting, scheduling, and sharing patient data information over a wireless network. The system includes a microprocessor which allows for layered administration access at the operator and medical center level, a memory module for receiving and securing patient data information, cloud storage in communication with the microprocessor, and a network application infrastructure which enables implementation on a computer or mobile computing device.

The system is configured to enable administrative access-granted medical centers and authorized personnel to receive a medical referral including the patient's medical information and to share amongst subscribers over a secure and wireless network. The network (i.e., platform) enables features such as report builders that allow specific data to be stored within the memory module and that are accessible to the user to build customer profiles with information such as insurance carriers, ICD10 codes, and the number of patients. The report builder will be accessible to any subscriber with the granted permission from the administrator and will enable data to be collected, disseminated, and shared amongst network users.

The system further provides secure patient communications using secure text and email notification and allows for specific “push notifications” to be transmitted over the network between a patient and subscribed medical center. Further, the system enables a plurality of pre-populated templates to be generated by an authorized user to fulfill a prescription and coordinate a delivery.

Moreover, in accordance with a preferred embodiment of the present invention, other aspects, advantages, and novel features of the present invention will become apparent from the following detailed description in conjunction with the drawings.

### BRIEF DESCRIPTION OF THE DRAWINGS

A more complete understanding of the present invention and the advantages and features thereof will be more readily understood by reference to the following detailed description when considered in conjunction with the accompanying drawings wherein:

FIG. 1 illustrates a schematic view of the control architecture of the system, according to an embodiment of the present invention;

FIG. 2 illustrates a block diagram of the system, according to an embodiment of the present invention;

FIG. 3 illustrates a screenshot of the user interface, according to an embodiment of the present invention; and

FIG. 4 illustrates a screenshot of the user interface, according to an embodiment of the present invention.

### DETAILED DESCRIPTION

The specific details of the single embodiment or variety of embodiments described herein are to the described system and methods of use. Any specific details of the embodiments are used for demonstration purposes only and not unnecessary limitations or inferences are to be understood therefrom.

Any reference to “invention” within this document is a reference to an embodiment of a family of inventions, with no single embodiment including features that are necessarily included in all embodiments, unless otherwise stated. Furthermore, although there may be references to “advantage's” provided by some embodiments, other embodiments may not include those same advantages or may include different advantages. Any advantages described herein are not to be construed as limiting to any of the claims.

Before describing in detail exemplary embodiments, it is noted that the embodiments reside primarily in combinations of components related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second” and the like, may be used solely to distinguish one entity or element from another entity or element without necessarily requiring or implying any physical or logical relationship or order between such entities or elements.

The system includes a central server 123 including a microprocessor 112 electrically connected to a memory module 116, and a user input/output medium which is wirelessly connected to a central server 128. The processor 112 is configured to collect, disseminate, and share a plurality of patient medical data information between a plurality of medical centers over a secure wireless network. The storage system may be any storage system such as local memory, fixed storage disks, remote storage disks, database, or otherwise configured to store secure patient medical data logs, shared medical information, insurance information, and medical center specific IC codes.

The server includes a first communications interface or other input/output port that is connected to a first communication network. A computer medium or smart device is equipped with a display and may be wired or wirelessly connected to the secure network. A hospital administrator or patient may import patient data information through an intake form or administrative controlled patient portal via a data transfer system or connection. The computer or smart device may respond to the customer I/O interface, which may be a patient or medical personnel located at a remote medical facility or doctor's office. The computer or smart device is configured with a patient medical data acquisition, compression, and transmission program for receiving patient medical data from a wired or wireless computer or smart device, for consolidating medical data information into a transferable file readily storable and transferable over the network.

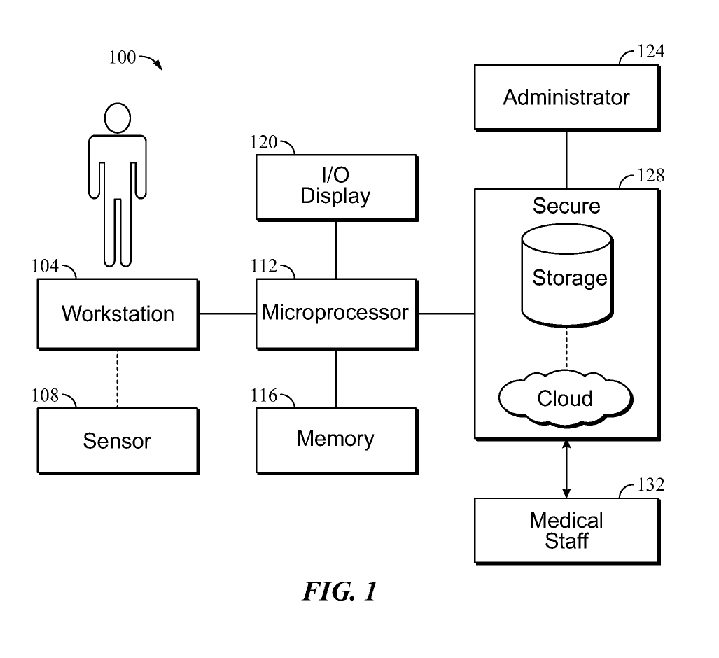

Referring now to the illustrations which depict the embodiments described, there is shown in FIG. 1 a system for collecting and collaborating medical data over a wireless network between a plurality of pre-designated in medical centers to enable effective patient management and scheduling. As shown in FIG. 1, the system receives patient data information from an input/output computer medium 120 or smart device which records the patient medical data and stores the patient medical data in a memory module 116 which is electrically connected to a central microprocessor 112. The memory module 116 may be local memory or may be a fixed or removable storage medium such as a flash card. The system provides a communication interface configured to download or transmit the patient medical data to the remote storage module such as a cloud-based storage medium.

For example, the communication interface between the interface and memory module may be a Universal Serial Bus (USB), Ethernet, Bluetooth, or transceivers commonly found in computer systems or smart devices. In such as arrangement, the remote computer includes a communication interface component which transfers the medical data to the data transfer system to the USB interface of the remote computer. The data transfer system may be any type for transferring data such as a serial interface, a parallel interface, a local area network interface, an infrared interface, an optical interface, or other hard-wired or wireless interface.

The data transfer system may also be a removable storage medium or mechanism for storage of the patient medical data such as a flash card. The remote computer system or smart device would each be configured for a parallel communication peripheral interface. Initially, the card would be able to store patient data. The patient medical data is removed, copied, or transferred by the remote computer or smart device for transfer to the cloud-based storage. Another memory storage devices to receive and contain patient medical data is available including floppy disks, a CD-ROM, a memory card/stick, or known readable memory mediums.

The remote computer or smart device segments the patient medical data and compresses the patient medical data transfer over the wireless network without affecting quality to ensure adequate transmission bandwidth. As patient medical data files are rather large files, which may be as large as 10 megabytes of data, the initial compression of the files is essential to ensure high-speed transmission.

The network may be any communication network capable of transmission of secure data. Some examples of this secure network may include any public service set identifier that broadcasts packet switched data. The network (i.e., Internet) is capable of supporting web pages and web page communications with large packet data information files. The remote computer or smart device equipped with an Internet communication interface or service from an Internet Service Provider (ISP) that enables communication of data through a central secure server which allows for data to be transceived from computers or smart devices.

The central server 128 hosts an administratively controller webpage hosted by a secure central server under the control of an administrative and authorized user who uploads and transmits the medical data and associated patient information from its memory to the central server to a designated medical center for intake and processing. This compressed and transmitted data may be selected using a variety of web page functions and drop down features which filter patient data information and hosted features. For example, it is contemplated that the webpage and system will allow data to be collected by a selected medical center, messaging will then be transmitted to the patient over the network including emails and push-notification which alert the patient of the medical center, authorized doctor, and time of the appointment.

A central server 128 connected to the network to accommodate the number of medical centers and authorized personnel using the system. In this case, the central server 128 hosts the webpage that is accessible by the computer or smart device through a variety of web browser. The computer or smart device used by the medical center, doctor, patient, or variety of authorized users transmit the patient medical data and patient information through a secure web portal on the webpage. The central server 128 transfers the patient medical data and patient information to the central server 128 over the network to an interface at a designated medical center. This contemplated configuration allows for more flexibility and hosting a plurality of Internet Service Providers (ISP) at a central hub in the first network, and the central server 128 may be remote at the designated medical center or patient's residence.

The central server 128, as common with any server, receives transmitted data over a network. The embodiments illustrated provide a server which facilitates and transmits patient data information between medical center/hospitals, and patients over a wireless network. In the embodiments, as many of the medical centers will have more than one single computer or smart devices, the central servers include a layer 128-bit configured server may be used for additional security.

The microprocessor 112 is configured to control access to and analysis of patient medical data using a central administrator 124 that has control access to medical data, reports with the report builders, etc. which is stored within the memory module 116 and includes audio and video files, uploaded photographs, spreadsheets, test data, doctor notes, charts, graphs, or other useful items.

The microprocessor 112 controls access to the patient data and any data transmitted to the central server 128 and may further store specific patient data information with a corresponding doctor, medical center, or hospital which is determined by the administrator. The microprocessor 112 further facilitates the medical center and patient notifications to the medical center and patient computer and smart devices that may receive and store patient data using the unique identifier at the customer portal of the web page.

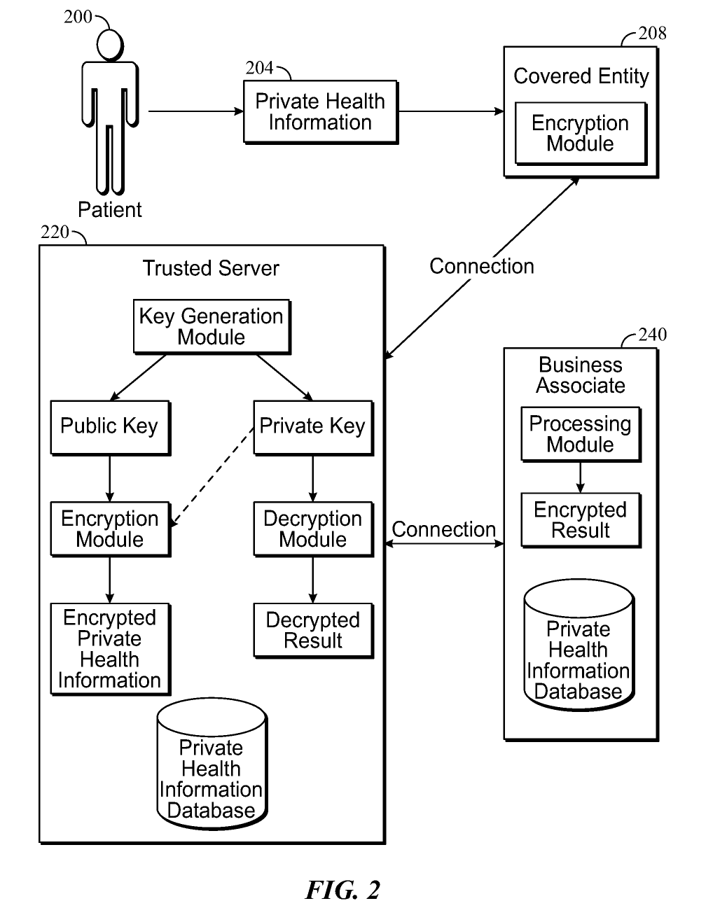

FIG. 2 illustrates one process for operating the system 100 according to the embodiment including accessing patient 200 information, patient medical data 204 and medical reports through the secure server 220. The system 100 allows the user to log into a secure server 220 which receives and stores patient information 204 from a patient, medical center, medical staff member, doctor, or hospital staff member 132 (i.e., authorized personnel). The server may correspond the received data to a specific patient 200 or plurality of patients including prior medical history and notes. The secure server 220 then allows for an authorized user to make additional comments or notes before being stored within the memory module 116 where they may be transferred to a third-party medical center or hospital.

Within the designated medical center a second authorized user 240 may log into the webpage after receiving a notification that the medical information was transferred. The authorized user may then select the uploaded medical data associated with at least one patient and transfer the information to a pre-selected department within the medical center using the secure server 220. The secure server 220 may restrict access to the medical data if specific permissions are not permitted by the administrator. For example, a surgical technician may not have the full medical data available to the surgeon assigned to the medical data at the hospital. After transferring the patient report at the server, the authorized user may annotate the medical report as being “under review” or “transferred” to a specific medical center. A further notification may be pushed to the patient or primary medical center of the progress of the appointment provided.

In some embodiments, the physician inputs a project identifier into the system 100 which recognizes the project tag and associates the project tag with a space. The system 100 directs the input to a display field in the associated project space. A secondary user logs in to the system 100 and accesses the project space to views and respond to input on the project tag. The system 100 recognizes the project tag and the app designates the physician whom input the project tag as the recipient of a reply. The system 100 transmits the reply to the desired display field in the project space and generates a notification for the reply. Once notifications are enabled by the physician, the physician accesses the project space and receives the notification of the reply.

When the medical procedure is completed with the patient, the patient medical center may be accessed through the web page and annotated as complete before being transmitted to the secure server 220. The secure server 220 receives and stores the medical report and any corresponding notes, charts, or similar files. The system further confirms if the feature is enabled to ensure a primary or secondary medical center is apprised of the patients care received and any follow appointment necessary. In an alternative embodiment, the computer or smart device is configured to access the web page using at least one web browser. In this configuration, the authorized user logs onto a secure server 220 through a secure portal where the patient medical data information is stored. If a referral or appointment at a further medical center is received, a report may be created to describe the patient's condition, recommended procedure, and any further pertinent medical information. A medical report is created and stored on the server, and it may be accessed by a designated medical center who receives an alert of a new matter. The secure server 220 then transmits the data to a dedicated medical center where it may be shared with at least one department for review.

Private health information 204 is transferred to a covered entity 208 and encrypted via encryption module 208. The information 204 is then transferred to the secure server 220 wherein information is encrypted/decrypted using encryption means known in the art. Encryption can be a public/private key having encryption and decryption modules in communication thereto. Information is then stored in the private health information database as described hereinabove. The second authorized user 240 can access information stored in the secure server 220 per administrative guidelines.

In one example, the key generation module of covered entity 208 generates a public key and a private key compatible with an asymmetric key encryption scheme. Once the public key and private key have been generated, the encryption module of covered entity fully homomorphically encrypts the private health information (and, if required by the applicable homomorphic encryption scheme, one or more instances of the private key) using at least one instance of the public key, yielding encrypted private health information. Accordingly, as used herein and in the claims, the terms “encrypted private health information” and “fully homomorphically encrypted private health information” may refer to only private health information that has been encrypted, or to both private health information in encrypted form and one or more instances of private key in encrypted form, as the context permits.

A decryption module of the covered entity 208 (or a secure server 202 if appropriate) decrypts the encrypted result using the private key, yielding a decrypted result. The covered entity can then make appropriate use of the decrypted result, such as using it for the benefit of the patient, for administrative purposes, or for improving the overall quality of the services provided by the covered entity.

In one embodiment, the patient 200 can choose to share medical information with any number of recipients via the network. This provides full access to appropriate healthcare professionals as well as the patient 200.

The invention is also a method for managing and scheduling medical referrals received by a designated medical center or medical specialist who is a subscriber of the system and who may download, open, and review the medical information shared. The downloaded medical staff may then be saved in the memory and later accessed by an authorized user.

In one embodiment, a method is presented Wherein a processor facilitates a process for sharing medical information and referrals. At least one user is authenticated and provided with information. Authentication can include an appropriate login credential submitted to the system. The information can be encrypted and decrypted to ensure security thereof. Communication means between the patient, and the medical professional can be provided. It is a goal of the system to facilitate a medical scheduling event as well as medical referrals, via the processor.

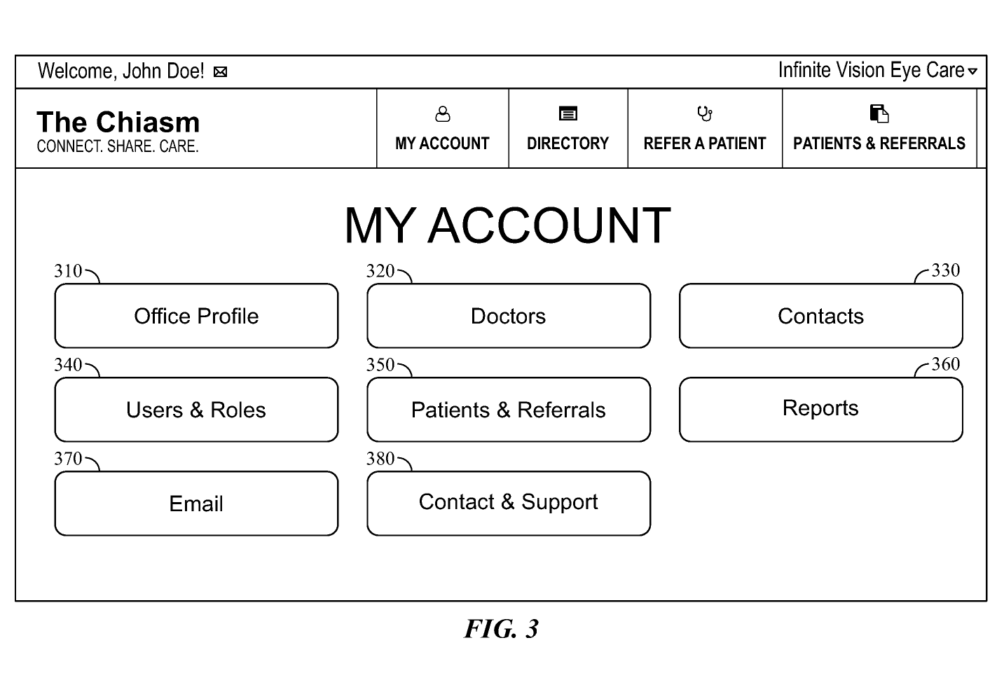

In reference to FIG. 3, a graphical user interface (GUI) is shown in one embodiment of the invention. Once a user, such as a healthcare professional, administrator, or similar entity accesses their account, the account interface 300 is shown having a variety of selectable interfaces thereon. In one example, selections can include but are not limited to, an office profile 310, doctor list 320, contacts 330, users and roles 340, patients and referrals 350, reports 360, email interface 370, and contact and support 380 selections.

The office profile 310 can include company/office information including names, locations, contact information and relevant information. The doctor list 320 can include a list of doctors utilizing the invention as described herein. Each medical professional on the list can include the name, location, contact information, specialty, and other information related to the medical professional or group thereof. New medical professionals may be added upon the request of the users within the network. The contacts list 330 can include any relevant contacts, whether businesses, individuals or otherwise.

A users and roles list 340 identifies users of the system and credentials thereof. This can include general users, administrators, or likewise users in the network. Similarly, the patients and referrals list 350 include a list of both patient referrals sent and received.

The reports list 360 provides a listing of referrals and appointments for the medical practitioners within the network. This provides a simple and organized listing of referrals and appointments both past and upcoming.

The email interface 370 and contact and support selection 380 provide further means of engagement within the system. The email interface 370 is in communication with a messaging mechanism to permit both patients and practitioners to contact one another.

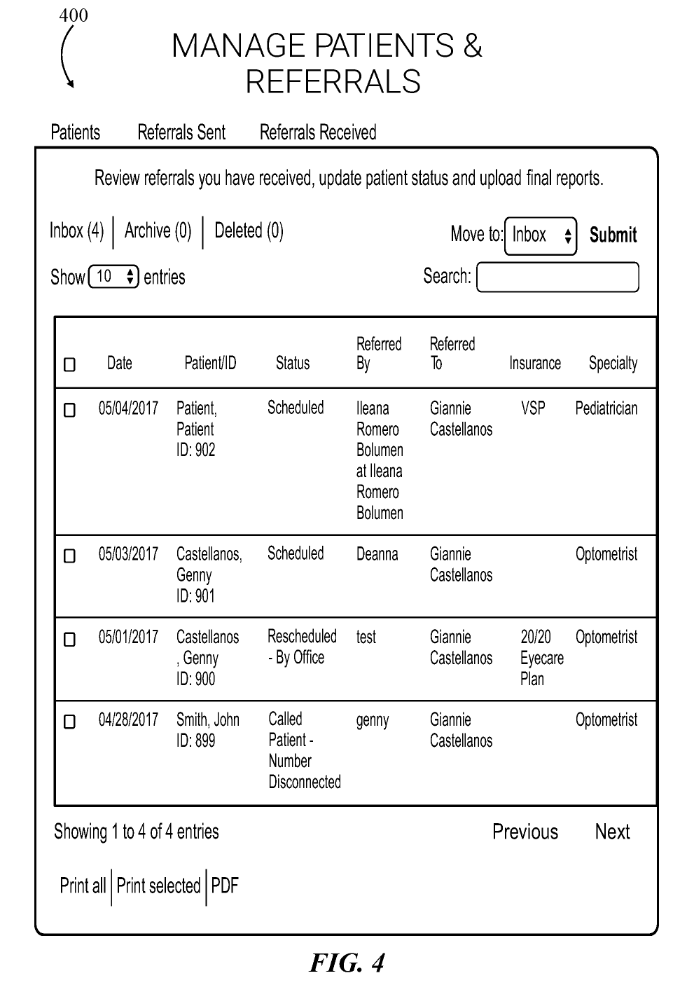

FIG. 4 illustrates a patient referral interface 400 having a plurality of referrals thereon. Information within interface 400 can include but is not limited to relevant dates, patient identifiers, status, referral references, patient insurance, and practice specialty. A search queue may be presented to permit searching of referrals and associated content thereof.

In one embodiment, the option to view sent and received referrals is presented.

One or more portions of the computer system may be distributed across one or more computer systems coupled to a communications network. For example, various aspects of the invention may be distributed among one or more computer systems servers) configured to provide a service to one or more client computers, or to perform an overall task as part of a distributed system. For example, various aspects of the invention may be performed on a client-server or multi-tier system that includes components distributed among one or more server systems that perform various functions according to various embodiments of the invention. These components may be executable, intermediate (e.g., IL) or interpreted (e.g., Java) code which communicate over a communication network (e.g., the Internet using a communication protocol (e.g., TCP/IP).

The computer system 100 also includes a plurality of input/output (“I/O”) devices 120 such as a keyboard, mouse, trackball, microphone, touch screen, printing device, display screen, speaker, among other I/O devices 120 commonly associated with the arts. In addition, the system 100 includes one or more network connection devices that connect the computer system to a communication network in addition to, or as an alternative to, the network.

A storage system includes a computer readable and writeable nonvolatile recording medium in which signals are stored that define a program to be executed by the processor 112 as well as information to be processed by the program installed thereon. In operation, the processor 112 causes data 204 to be read from the nonvolatile recording medium into another memory that allows for faster access to the information by the processor than does the medium. This memory is a volatile, random access memory such as a dynamic random access memory (DRAM) or static memory (SRAM). The memory 116 may be located in the storage system, as shown. The processor 112 generally manipulates the data within the memory 116 and then copies the data to the medium associated with storage after processing is completed. A variety of mechanisms are known for managing data movement between the medium and integrated circuit memory element, and the invention is not limited thereto. The invention is not limited to a particular memory system or storage system. In one example, content may be stored on computer-readable medium, as well as graphical indicators for content. Targeting criteria may be stored as well, among other options. The processor 112 and operating system together define a computer platform for which application programs in high-level programming languages are written. It should be understood that the invention is not limited to a particular computer system platform, processor, operating system, or network. Also, it should be apparent to those skilled in the art that the present invention is not limited to a specific programming language or computer system. Further, it should be appreciated that other appropriate programming languages and other appropriate computer systems could also be used.

System 100 may include a server process that responds to requests from one or more client programs. The process may include, for example, an HTTP server or other server-based processes (e.g., a database server process, XML server, peer-to-peer process) that interfaces to one or more client programs distributed among one or more client systems.

In some embodiments, the system 100 is in operable communication with a third-party system in communication with the second authorized user 240. This third-party provides patient insurance information, such as patient coverages to the system 100. The system 100 can include a comparator to compare coverage plans and insurance plans between insurance providers.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A system for scheduling and collaborating medical information over a network, the system comprising:
a. a microprocessor electrically connected to a memory module and wirelessly connected to a secure server for receiving and collaborating patient data information to a plurality of subscribed medical centers;
b. a memory module configured to receive and store a plurality of transmitted medical information corresponding to a patient referred to a pre-determined medical center for a medical procedure at a time; and
c. a database server configured to a share a plurality of medical data amongst a plurality of administrative controlled authorized users to facilitate medical scheduling and referrals.

2. The system of claim 1, further including a messaging mechanism which enables a plurality of updates to be sent using a notification means to a patient and the at least one subscribed medical center.

3. The system of claim 1, wherein the secure server further includes a cloud-based server which enables a plurality of shared data within a report database to be accessed.

4. The system of claim 3, further including a transportation module to coordinate transportation through a third-party provider between at least one patient and the subscribed medical center.

5. The system of claim 1, further comprising a third-party system configured to determine patient insurance plans.

6. The system of claim 1, further comprising a user interface accessible by a healthcare professional, wherein the user interface displays the plurality of shared medical data.

7. The system of claim 6, wherein the microprocessor performs the following:
a. authenticating at least one user;
b. providing information to the user;
c. encrypting and decrypting the plurality of shared medical data;
d. providing communication means via a network;
e. facilitating medical scheduling; and
f. facilitating medical referrals.

8. The system of claim 1, wherein the secure server is comprised of a key generation module configured to generate a public key and a private key to encrypt the plurality of shared medical data, wherein a private health information database stores the plurality of encrypted shared medical data.

9. The system of claim 1, wherein each of the plurality of shared data is associated with a patient, wherein the plurality of shared data is accessible by the healthcare professional and the associated patient.

10. A system for scheduling and collaborating medical information over a network, the system comprising:
a. a microprocessor electrically connected to a memory module and wirelessly connected to a secure server for receiving and collaborating patient data information to a plurality of subscribed medical centers, wherein the memory module is configured to receive and store a plurality of transmitted medical information corresponding to a patient referred to a pre-determined medical center for a medical procedure at a time;
b. a database server configured to a share a plurality of medical data amongst a plurality of administrative controlled authorized users to facilitate medical scheduling and referrals;
c. a processor configured to perform the following:
i. authenticating at least one user;
ii. providing information to the user;
iii. encrypting and decrypting the plurality of shared medical data;
iv. providing communication means via a network;
v. facilitating a medical scheduling event; and
vi. facilitating medical referrals.

11. The system of claim 10, further including a messaging mechanism which enables a plurality of updates to be sent using a notification means to a patient and the at least one subscribed medical center.

12. The system of claim 10, wherein the server further includes a cloud-based server which enables a plurality of shared data within a report database to be accessed.

13. The system of claim 12, further including a transportation module to coordinate transportation through a third-party provider between at least one patient and the subscribed medical center.

14. The system of claim 10, wherein the system further provides a pre-populated template between a subscribed medical center and a pharmacy to allow for a pharmacy to be fulfilled.

15. The system of claim 10, wherein the secure server is comprised of a key generation module configured to generate a public key and a private key to encrypt the plurality of shared medical data, wherein a private health information database stores the plurality of encrypted shared medical data.

16. The system of claim 10, wherein each of the plurality of shared data is associated with a patient, wherein the plurality of shared data is accessible by the healthcare professional and the associated patient.

17. The system of claim 10, further comprising a verification module configured to determine the eligibility of a user to view the plurality of shared medical data.

18. The system of claim 17, wherein the verification module, via the processor, performs the following:
a. assigning credentials to each user;
b. receiving a request to engage with information;
c. determining if the request is appropriate for the specific user;
d. accepting or denying the request;
e. displaying information to the specific user.

19. The system of claim 18, further comprising a third-party system configured to determine patient insurance plans.

20. The system of claim 10, further comprising the step of a user, via the user interface, adding a new medical center, a new patient, and a new medical professional to the database.
